(19) United States
(12) Patent Application Publication
(10) Pub. No.: US 2005/0048569 A1
(43) Pub. Date: Mar. 3, 2005

(54) Method of clustering transmembrane proteins

(76) Inventors: Petrus Johannes Van Der Spek, Lille (BE); Maroesja Maria Jannetje Van Nimwegen, Leiden (NL); Jean-Marc Edmond Fernand Marie Neefs, Lier (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/499,955

(22) PCT Filed: Dec. 20, 2002
(86) PCT No.: PCT/EP02/14868

(30) Foreign Application Priority Data
Dec. 21, 2001 (EP) 01205090.2

(51) Int. Cl. G01N 33/53; G06F 19/00; G01N 33/48; G01N 33/50

(52) U.S. Cl. 435/7.1; 702/19

(57) ABSTRACT

A method and apparatus for clustering polypeptide sequences, and in particular transmembrane proteins, is disclosed. Intra-membrane regions are isolated and the amino acid labels replaced with one or more physical/chemical parameters. The resulting data vectors are analysed using a clustering technique based on correlation between the data vectors, for example using aglomerative hierarchical clustering.

The present invention relates to the clustering of transmembrane proteins so as to identify similar or functionally related sequences, and in particular, but not exclusively, to the clustering of G-protein coupled receptors.

Signalling of a wide variety of ligands including Ca⁺, odorants, light, amino acids, nucleotides, peptides and hormones is mediated through GTP-binding protein (G-protein)-coupled receptors (GPCRs). These GPCRs represent the largest family of cell-surface molecules involved in signal transduction in eukaryotes and certain prokaryotes. The characteristic motif of this superfamily of plasma membrane bound receptors is the seven hydrophobic regions that are collectively known as a transmembrane (tm) domain. Of the 800 GPCRs that are cloned to date, for a group of them, the ‘orphan’ receptors, the ligand still has to be identified.

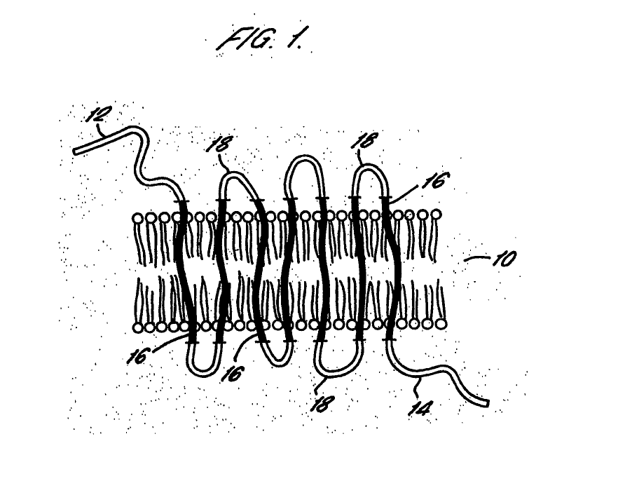

A GPCR is schematically illustrated in FIG. 1, positioned within a cellular membrane 10. The GPCR consists of three different domains, an extra-membrane N-terminal 12 or extracellular domain, a extra-membrane C-terminal 14 or intracellular domain and a transmembrane domain. The transmembrane domain consists of 7 intra-membrane regions 16 linked by extra-membrane loops 18. The intra-membrane regions show very high sequence conservation, whereas the extracellular domains, intracellular domains and the intervening loops show low sequence conservation. The transmembrane domain is not only conserved between GPCRs, but also between species which is useful to identify functional equivalents in model organisms. The N-terminal domain is of variable size and is involved in ligand binding, activation and down-regulation of the GPCR, whereas the C-terminal domain is responsible for the activation of a class of G-proteins. The 7 helices of the transmembrane domain are thought to be arranged as a tight, ring-shaped core. Hydrophobic amino acid residues are most likely to be located near the lipid bilayer, whereas hydrophilic amino acid residues face the centre of the membrane. Helix-helix interactions of the 7 helices are responsible for the tertiary structure of the GPCR and thereby important for receptor folding and stability, ligand binding and ligand-induced conformational changes for G-protein coupling.

GPCRs are very interesting targets for developing new drugs since they play key roles in a wide range of diseases, their expression is tissue specific and their function can be agonized or antagonized by small molecules. The agonists and antagonists are not yet known for all GPCRS. However by grouping GPCRs of known function with those which are less well understood, it is possible to deduce biochemical functionality of the less understood GPCRs to thereby identify potential new drug targets. Known methods of grouping GPCRs and other polypeptides rely on statistical comparisons between the amino acids in groups of aligned sequences, which is not always very effective. It would therefore be desirable to provide an improved method of comparing and grouping GPCRs.

Other transmembrane proteins such as ion channel and glycotransport proteins also comprise a number of extra-membrane and intra-membrane regions, and, advantageously, could also be grouped using such an improved method of grouping so as to identify relationships between the proteins.

Hobohm and Sander (Journal of Molecular Biology 1995, 251, 390-399) sought to define protein sequence dissimilarity as a weighted sum of differences of compositional amino acid properties such as singlet and doublet amino acid compositions, molecular weight, isoelectric point, and aliphatic, aromatic, polarity, size and charge properties. An algorithm was used to determine the optimal weight to be given to each property in order to best distinguish between 58 selected protein families.

Sandberg et al. (Journal of Medical Chemistry 1998, 41, 2481-2491) derived an improved set of amino acid z-scales. Each z-scale is a different combination of experimental and calculated amino acid properties, and the set of z-scales is optimized to distinguish between different amino acids for the purposes of quantitative structure-activity modelling.

In summary, the invention provides a method of grouping a number of related protein sequences, and especially a number of related transmembrane proteins such as GPCRs. This is carried out by isolating one or more equivalent domains, such as transmembrane domains or inter-membrane regions, in each of the related sequences, substituting the amino acids in each domain with one or more physical/chemical amino acid properties such as molecular weight or hydrophobicity, and applying a clustering or grouping analysis to the resulting sets of physical/chemical properties. It is found that functionally related transmembrane proteins are grouped together much more effectively when using this method than when simply applying a clustering or grouping analysis directly to the amino acid sequences.

According to one aspect the present invention provides a method of grouping, ordering, clustering or otherwise logically arranging a plurality of transmembrane protein sequences, each protein sequence comprising one or more intra-membrane regions and one or more extra-membrane regions, comprising the steps of: forming a set of amino acid labels for each protein sequence, each set including a plurality of amino acid labels from one or more of said intra-membrane regions and excluding at least some of the amino acid labels from said extra-membrane regions, each amino acid label corresponding to a positionally equivalent amino acid label in each of the other sets of amino acid labels; forming a set of physical/chemical properties for each set of amino acid labels by substituting one or more different physical/chemical amino acid properties for each amino acid label; and grouping, ordering, clustering or otherwise arranging the sets of physical/chemical properties.

Intra-membrane and extra-membrane, or transmembrane regions, can generally be identified with corresponding hydrophobic and hydrophilic regions. Each set may advantageously be provided by a data structure in a computer, which is programmed to carry out at least the substitution of amino acid properties and the step of grouping.

The amino acid labels will typically be the alphabetic codes conventionally used for amino acids, i.e. “A” for Alanine, “C” for Cysteine and so on, although any suitable labelling scheme, including schemes which use a single label for two or more amino acids which are similar in one or more respects may be used. Each amino acid label should correspond to a positionally equivalent amino acid label in each other set of labels so that each particular amino acid from a first of the sequences, when converted to one or more physical/chemical properties, can be compared directly with the corresponding amino acid in each of the other sequences. Just one type of physical/chemical property may be used in place of each amino acid label, or several different properties may be used, and it is not essential for all labels to be translated, into all of the property types used as long as the same property types are used for each positionally equivalent amino acid label in each set.

By omitting some or all of the amino acid labels of the extra-membrane regions the quality of the groupings generated, from a biological perspective, is improved. Preferably, therefore, each set of amino acid labels excludes substantially all of the amino acids from the extra-membrane regions.

Conversely, to obtain the most biologically useful results, as much of the intra-membrane regions as possible should be included in the grouping analysis. Preferably, therefore, each set of amino acid labels includes substantially all of the amino acid labels from the intra-membrane regions.

The physical/chemical characteristics used to establish the sets of amino acid properties may be selected from a list comprising molecular weight, hydrophobicity, hydrophilicity, surface area and isoelectric point. Measures of dissociation or acidity, such as pKA, may also be used, as may a variety of other conventionally used amino acid characteristic properties familiar to the person skilled in the art. Suitable physical/chemical properties may also be provided by combining several experimental and/or calculated properties, such as those described above, in predetermined ways, for example the z-scales discussed in Sandberg et al. (Journal of Medical Chemistry 1998, 41, 2481-2491). Preferably, several of the chosen properties are used simultaneously for each protein sequence.

A variety of statistical methods may be used to carry out the step of grouping, such as aglomerative or divisive clustering schemes known to the skilled person.

Preferably, numerical correlation between the sets of physical/chemical properties or clusters of such properties is used as a distance measure in the step of grouping, although other distance measures, for example based on Euclidean distance, could be used.

A similar method may be used on other polypeptide sequences which do not arise in transmembrane proteins. Accordingly, the invention also provides a method of grouping a plurality of polypeptide sequences comprising the steps of: forming a set of amino acid labels from each polypeptide sequence, each amino acid label corresponding to a positionally equivalent amino acid label in each of the other sets of amino acid labels; forming a set of physical/chemical properties from each set of amino acid labels by substituting each amino acid label with one or more physical/chemical amino acid properties; and grouping the sets of properties so as to identify groupings of said polypeptide sequences.

In practice, the above methods are carried out using a suitably programmed computer, and the steps of the methods may be embodied in computer program elements which may be written on suitable computer readable media. Accordingly, the invention also provides an apparatus for clustering a plurality of transmembrane protein sequences, to thereby aid identification of relationships between said transmembrane protein sequences, each sequence comprising one or more intra-membrane regions and one or more extra-membrane regions, comprising:
a segmentor arranged to form a set of amino acid labels from each protein sequence, each set including a plurality of amino acid labels from one or more of said intra-membrane regions and excluding at least some amino acid labels from said one or more extra-membrane regions, each amino acid label corresponding to a positionally equivalent amino acid label in each of the other sets of amino acid labels;
a translator arranged to form a set of physical/chemical properties from each set of amino acid labels by substituting each amino acid label with one or more physical/chemical properties; and
an analyser arranged to cluster or order the sets of physical/chemical properties.

Embodiments of the invention will now be described, by way of example only, with reference to the accompanying drawings, of which:

FIG. 1 schematically illustrates the intra-membrane and extra-membrane regions of a G-protein coupled receptor sited in a cellular membrane;

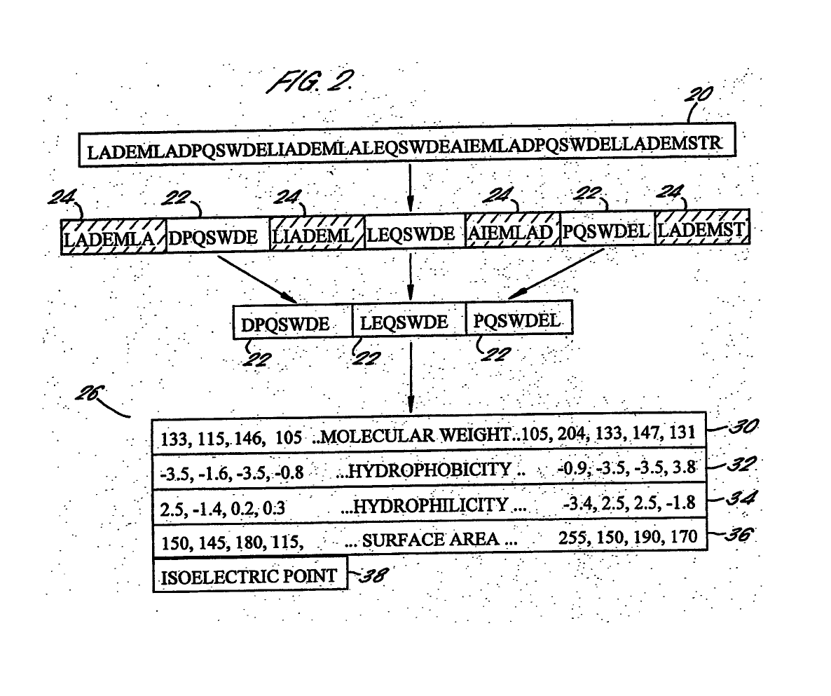

FIG. 2 illustrates steps of the method of the preferred embodiment;

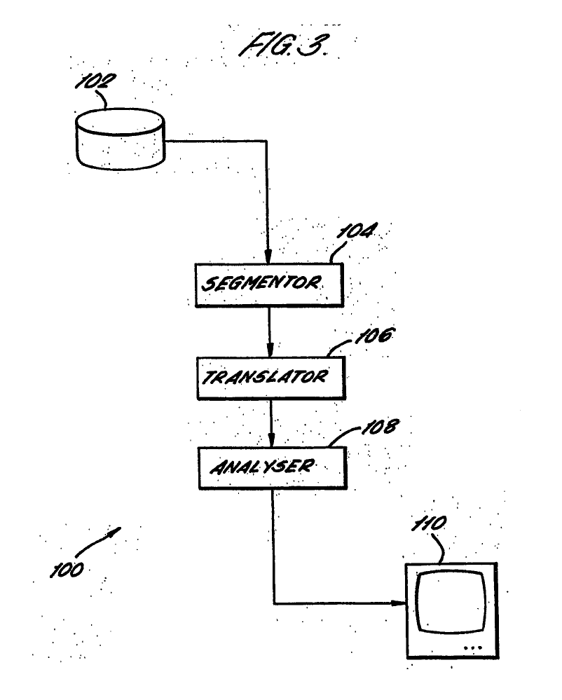

FIG. 3 shows, schematically, apparatus for carrying out the method of FIG. 2;

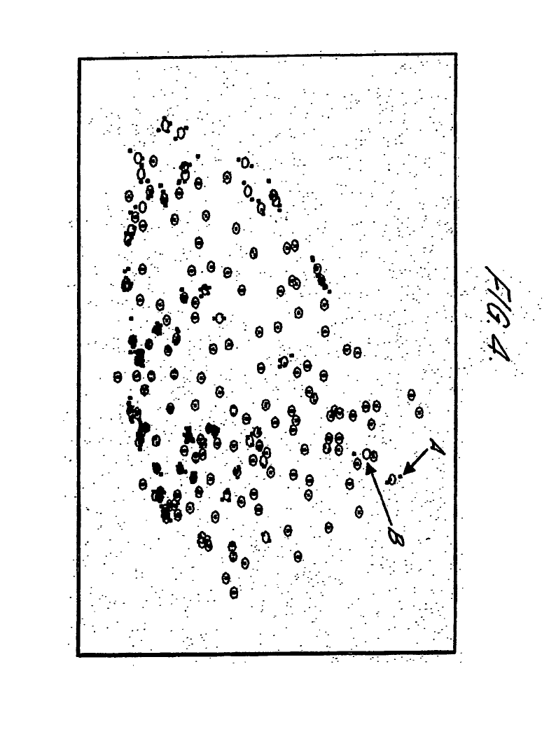

FIG. 4 is a proximity plot of human GPCR sequences (dots) and clusters of these GPCRs (ovoids) following processing of GPCR data according to the method illustrated in FIG. 2;

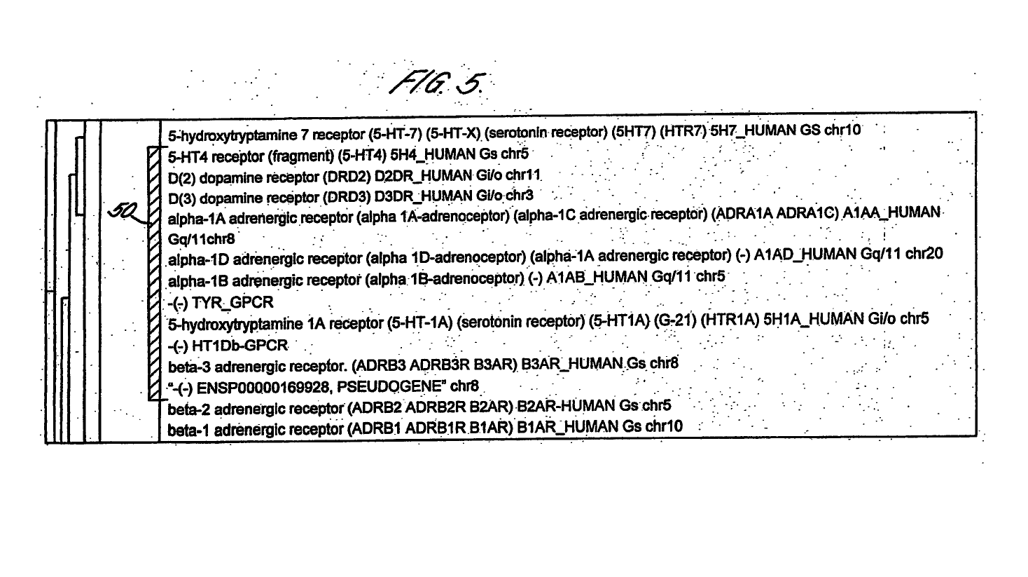

FIG. 5 provides details of a cluster obtained using the method of FIG. 2, containing predominantly dopaminergic and adrenergic GPCRs;

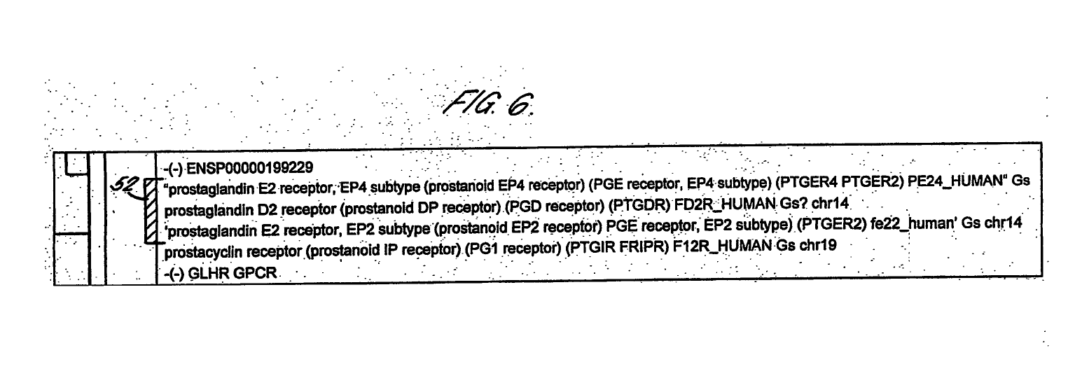

FIG. 6 shows a cluster obtained using the method of FIG. 2, containing only prostaglandin receptors;

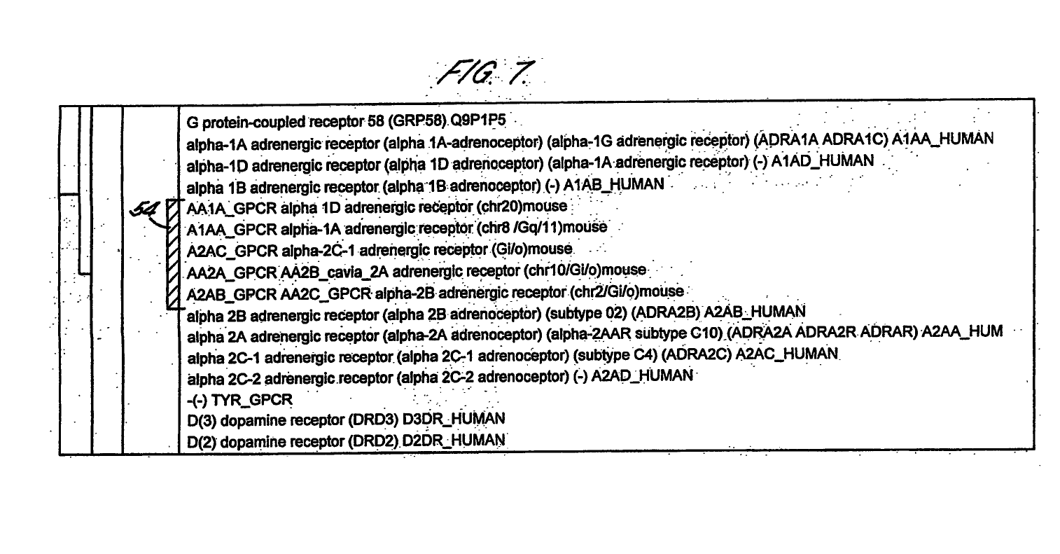

FIG. 7 shows a cluster containing mouse adrenergic GPCRs clustered together with their human orthologues using the method;

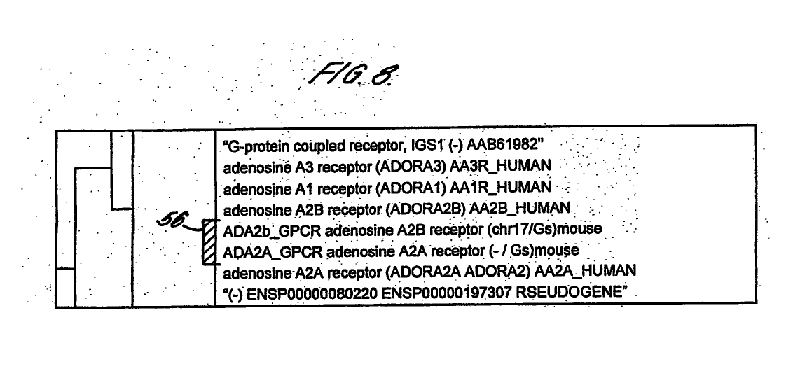

FIG. 8 shows adenosine mouse GPCRs clustered together with human adenosine GPCRs using the method;

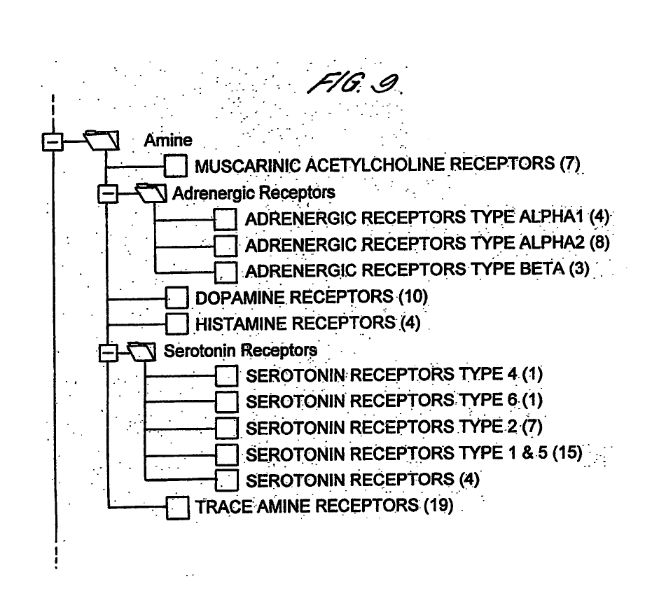

FIG. 9 shows amine-type receptors according to a published categorisation; and

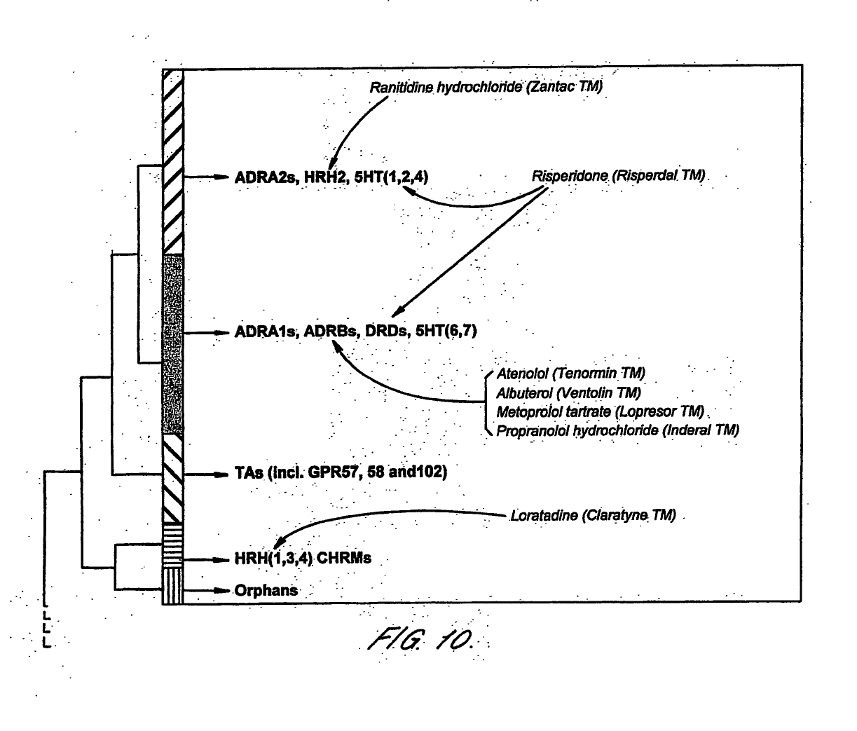

FIG. 10 shows amine-type receptors ordered using the method.

A described embodiment of the invention is a method of clustering a plurality of related transmembrane proteins, the method consisting of steps of collecting together and aligning with each other the protein sequences, isolating the intra-membrane regions of the protein sequences, translating the amino acid names of the intra-membrane regions into sequences of physical/chemical properties and carrying out a clustering or grouping exercise on the property sequences. The results of the clustering exercise can then be used to deduce likely biological and biochemical relationships within the plurality of related proteins, so that less well characterised proteins can be better understood with reference to the better understood proteins. This method will now be described with reference to FIG. 2, which shows the processing of a single protein sequence 20.

An appropriate plurality of transmembrane proteins 20 may be collected together using techniques familiar to the skilled person, in particular by making use of publically available databases. Typically, one or more well characterised transmembrane proteins are used as target sequences in an alignment exercise against available databases of polypeptide or polynucleotide data to find other sequences which are sufficiently similar. To carry out the method of the preferred embodiment, it is necessary to ensure that the intra-membrane regions 22, which are usually characterised by hydrophobic helix segments, are well aligned between the protein sequences, so as to establish a one-to-one relationship between each of the amino acids of these regions. The intra-membrane regions 22 of each protein sequence are then isolated to form a set of amino acid labels or names 26. Each set includes the intra-membrane 22 but excludes the extra-membrane amino acids 24 and there is a one-to-one correspondence, based on equivalent positions in the original protein sequences, between members of the sets, which are consequently all of the same length. The precise divisions between intra and extra membrane regions (22, 24) is not of importance, as long as the same division is used for all of the protein sequences. Conveniently, the divisions may be determined with reference to publically available data for providing precise annotations of the relevant regions of one or more of the proteins.

The sets of amino acid names are used to form corresponding sets of physical/chemical properties 26. Each set of physical/chemical properties corresponds to one of the proteins, but may be made up of two or more series (30, 32, 34, 36, 38), each series comprising the same set of amino acid names converted into a different physical/chemical properties.

Each amino acid name is translated into one or more physical/chemical properties with reference to information such as that set out in table 1, which provides molecular weight, hydrophobicity, hydrophilicity and accessible surface area values for each type of amino acid. Such tables are commonly found in the prior art.

TABLE 1

| | | | | Accessible |
|---|---|---|---|---|
| | Molecular | Hydro/ | Hydro/ | surface |
| Amino acid | weight | phobicity | philicity | area |
| Alanine (A) | 89.1 | 1.8 | −0.5 | 115 |
| Cysteine (C) | 121.2 | 2.5 | −1.0 | 135 |
| Aspartate (D) | 133.1 | −3.5 | 2.5 | 150 |
| Glutamate (E) | 147.1 | −3.5 | 2.5 | 190 |
| Phenylalanine (F) | 165.2 | 2.8 | −2.5 | 210 |
| Glycine (G) | 75.1 | −0.4 | 0.0 | 75 |
| Histidine (H) | 155.2 | −3.2 | −0.5 | 195 |
| Isoleucine (I) | 131.2 | 4.5 | −1.8 | 175 |
| Lysine (K) | 146.2 | −3.9 | 3.0 | 200 |
| Leucine (L) | 131.2 | 3.8 | −1.8 | 170 |
| Methionine (M) | 149.2 | 1.9 | −1.3 | 185 |
| Asparagine (N) | 132.1 | −3.5 | 0.2 | 160 |
| Proline (P) | 115.1 | −1.6 | −1.4 | 145 |
| Glutamine (Q) | 146.2 | −3.5 | 0.2 | 180 |
| Arginine (R) | 174.2 | −4.5 | 3.0 | 225 |
| Serine (S) | 105.1 | −0.8 | 0.3 | 115 |
| Threonine (T) | 119.1 | −0.7 | −0.4 | 140 |
| Valine (V) | 117.1 | 4.2 | −1.5 | 155 |
| Tryptophan (W) | 204.2 | −0.9 | −3.4 | 255 |
| Tyrosine (Y) | 181.2 | −1.3 | −2.3 | 230 |

The set of physical/chemical properties 26 shown in FIG. 2 consequently comprises a series of molecular weight values 30, a series of hydrophobicity values 32, a series of hydrophilicity values 34, and a series of accessible surface area values 36.

Also shown is a series of isoelectric point values 38. However, whereas the other series each contain one property value for each amino acid in the set 24, the isoelectric point is calculated as a single value for each intra-membrane region 22.

In an alternative embodiment, optimized combinations of particular experimental and/or calculated physical/chemical properties may be used, such as the z-scales discussed in Sandberg et al, (Journal of Medical Chemistry 1998, 41, 2481-2491). Such z-scale type physical/chemical properties seek to represent the functional behaviour of amino acids in an optimal manner using a minimum number of derived physical/chemical properties. Sandberg et al. determine five optimised z-scale variables for representing amino acids.

Each set of physical/chemical properties 26 may be considered as a single vector of numbers, each number in each vector being directly comparable to the corresponding number in each other vector. Thus the sets of physical/chemical properties can be grouped using conventional vector clustering tools. In the preferred embodiment, aglomerative hierarchical clustering is used, although various other clustering schemes could equally be used, such as divisive clustering schemes. Aglomerative hierarchical clustering starts with each vector being considered as a separate cluster. The two most similar clusters are then joined together to form a larger cluster, and this step is repeated until the total number of clusters is reduced to below a threshold, or to one. The sequence in which the clustering takes place defines a tree structure which may conveniently be used to provide a graphical representation of the results of the clustering exercise.

To determine the similarity between two vectors of physical/chemical property values a correlation between the two vectors is used. This is straightforward to carry out in the present embodiment because all the sets of physical/chemical properties can be represented as vectors of the same length. However, to balance the contribution to the similarity measure of the different physical/chemical property values, which generally have different ranges of magnitude, appropriate weighting factors are used for the different property types. To determine the similarity between two clusters of vectors or a vector and a cluster, during the aglomerative clustering process, a simple geometric centroid of each cluster or other suitable mean may be used.

Another grouping technique used in the preferred embodiment to provide a different view of the data is a principal component analysis, from which a two dimensional proximity map can be formed and graphically displayed. Whether the results of grouping are displayed as a proximity map or as a tree, information such as name and known characteristics regarding each protein is made available graphically in association with the grouping data, so that inferences can be rapidly drawn from the displayed groupings.

Apparatus 100 adapted to carry out the methods described above is illustrated in FIG. 3. A database 102, or a plurality of databases stores protein sequence data from which the chosen transmembrane proteins are selected and forwarded to segmentor 104. The segmentor 104 carries out at least the process of isolating the intra-membrane regions of the protein sequences, and may also carry out alignment of the sequences if this has not been done prior to storage in the database 102.

The corresponding sets of amino acid labels from each intra-membrane region isolated by the segmentor are forwarded to a translator 106 where the amino acid labels are substituted for physical/chemical properties. The results of the substitution are forwarded to an analyser 108 which carries out the clustering processes in which the protein sequences are ordered, or the vector space defined by the sets of physical/chemical properties is collapsed in a manner such that the sequences are grouped together in associated clusters or can easily be visualised as such using a graphical display. The results of the processing carried out by the analyser 108 may then be displayed graphically on a visual display 110.

The apparatus 100 may conveniently be effected by means of a suitably programmed personal computer or workstation. The database 102 may be implemented, for example, on a storage medium local to the workstation or accessed over a network. Typically, the usual input and output devices such as a computer mouse, keyboard and visual display 110 will be provided to enable a user to control the apparatus.

More specific examples of the preferred embodiment will now be presented. The first example relates to an analysis, embodying the invention, carried out on a set of human GPCRs. To select all human GPCRs, a PSI-BLAST alignment exercise was performed using as template sequences a set of known GPCRs (from SWISS-PROT, TREMBL and ENSEMBL-pep) against several public and patented or proprietary protein sequence databases (Incyte Lifeseq®,DGENE, SWISSPROT, TREMBL, ENSEMBL). By removing duplicate and orthologue result sequences the number of GPCRs found with the alignment exercise was reduced. The latter process was performed in part manually and in part by sequence alignment using CLUSTAL-W.

To isolate the intra-membrane regions the GPCR sequences were aligned to a reference set of characterized GPCRs from GPCRdb (Gerrit Vriend, University of Nijmegen, The Netherlands). This reference set included only GPCRs with precisely annotated domains (extracellular and intracellular domains, intra-membrane regions, intervening loops). To ensure inclusion of the whole of the intra-membrane regions, for each GPCR three amino acids of the extracellular and three amino acids of the intracellular intervening loops were added to the isolated intra-membrane regions. The seven isolated intra-membrane regions together comprised 225 amino acids for all of the GPCRs.

The amino acid names in each set of intra-membrane regions were converted into values for hydrophobicity, hydrophilicity, accessible surface area and molecular weight, using the values set out in table 1. Additionally, the isoelectric point was calculated for each intra-membrane region using the ISOELECTRIC program of the GCG sequence analysis software suite v10.2. These physical/chemical property values were used to construct a data vector for each GPCR.

The data vectors were imported into Omniviz (RTM) data and visualization software. In order to obtain equal weight of the different physical/chemical parameters, the isoelectric point values were repeated a total of 32 times in each data vector, whereas the other values were used only once. Each data vector thus comprised a total of 1124 values for each GPCR (225 hydrophobicity values, 225 hydrophilicity values, 225 molecular weight values, 225 surface area values and 224 (32×7) isoelectric point values). The GPCR data vectors were hierarchically clustered on all 1124 values equally into 170 groups using the Omniviz (RTM) software, using an aglomerative hierarchical clustering scheme.

Each of the 170 cluster groups established by the Omniviz (RTM) software contained various numbers of GPCRs, ranging from 1 to 129. The GPCRs and groups are shown as dots and ovoids respectively in FIG. 4, which was generated using a principal component analysis supported by a number of heuristics to reduce the data space into a useful two dimensional proximity map.

The results of the clustering analysis were also displayed using the “Treescape” display function of the Omniviz (RTM) software. In this display mode, each row of the display represents a different GPCR. In a first area of the display a tree structure illustrates the structure of the bifurcating tree generated by the aglomerative hierarchical clustering, while other areas of the display contain color coded blocks representing each physical/chemical parameter. In an alternative “Treescape” display mode textual information identifying names and functions of characterised GPCRs are shown alongside the tree.

Using the Treescape display it was seen that GPCRs known to be related in function were clustered together. For example, one cluster (50) contained predominantly dopaminergic and adrenergic GPCRs (FIG. 5) whereas a cluster (52) in another part of the tree only contains prostaglandin receptors (FIG. 6). The groups did not only contain annotated GPCRs. Orphan GPCRs (GPCRs with an unknown function and/or an unknown ligand) clustered in groups together with the annotated GPCRs. Because of the ability to cluster orphan GPCRs together with annotated GPCRs instead of clustering with only other orphan GPCRs, the method can be used to predict the function or the identification of novel ligands for orphan GPCRs.

To provide further evidence that the ‘orphan’ GPCRs had a function and/or ligands that were related to the GPCRs in their cluster, 7 mouse orthologues were added to the human GPCR dataset discussed above. Three of the added mouse GPCRs were adrenergic receptors and the other four were adenosine GPCRs. Clustering of this new dataset of 746 human with 7 mouse GPCRs resulted in mixed clusters of human and mouse GPCRs. Mouse adrenergic GPCRs clustered together with their human orthologues, as shown by cluster 54 in FIG. 7. The adenosine mouse GPCRs that were added to the dataset clustered together with the human adenosine GPCRs, as shown in cluster 56 in FIG. 8.

In the second example, sequences of known or putative GPCRs were selected from public or proprietary databases. These sequences were of human origin unless no human orthologue was available. For each of the sequences, the 7 transmembrane regions were identified. For each transmembrane region, the isoelectric point was calculated. For each amino acid within these regions, four physical/chemical properties were calculated: hydrophilicity, hydrophobicity, molecular weight and surface area. This whole data set was analysed using OmniViz (TM). Hierarchical clustering of the GPCRs based on the 5 physical/chemical properties of the amino acids resulted in several homogenous clusters.

To evaluate the clustering results a classification of known GPCRs into functional subfamilies was retrieved from a public GPCR resource (http://www.gpcr.org/7tm/). FIG. 9 illustrates the GPCRs assigned by the classification as amine type receptors. The number of GPCRs in each subgroup is shown in parenthesis after the subgroup name. The clustering method grouped closely together 76 of the 83 amine type receptors. All of the remaining 7 amine type receptors are considered to be poorly understood and may well be wrongly classified as amine type receptors.

FIG. 10 illustrates, using the same clustering tree format as used in FIGS. 5 to 8, the clustering of some of the sub families of amine type receptors effected using the above method. The mapping of some commercial drugs onto the GPCRs is also shown. For some of the amine type GPCRs, the clustering can be observed down to subtype level. For example, the alpha adrenergic receptors 1 and 2 are accurately divided. Also the histamine H2 receptor is divided from the other histamine receptors.

The results of the clustering method were also compared with experimental results and conclusions found in the related scientific literature.

It has been shown that UDP-glucose is a potent agonist of the human orphan GPCR KIAA0001 (Freeman et al., 2001, Genomics, 78, 124-128). Of 45 GPCRs which are clustered closest to this orphan using the above method, the ligand is unknown for 22. Of the remaining 23 classified GPCRs, 10 belong to the (putative) purinergic receptors and 8 are peptide binding (angiotensin, bradykinin, chemokine, etc).

Kojima et al. 2000 (Biochemical & Biophysical Research Communications, 276, 435-438) identified the endogenous ligand for GPR66 as being neuromedin U. Using the clustering method described above, GPR66 belongs to a small cluster, together with three other GPCRs. From these 4 GPCRs, only one has been well annotated and classified: neuromedin U. This cluster is in immediate vicinity to other neuropeptide binding GPCRs.

Lin et al. have submitted an article to the Journal of Biochemistry indicating that the ligand for GPR73 is prokineticin. This GPC clusters closest to galanin receptors. This is in contrast to the clustering closest to neuropeptide receptors in a conventional phylogenetic tree. Prokineticin is thought to play a role in GI smooth muscle contraction. Galanin contracts smooth muscle of the GI and genitournary tract, regulates growth hormone release, modulates insulin release and may be involved in the control of adrenal secretion. Hence, the close clustering of GPR73 to galanin receptors is very plausible.

In the third example the GPCRs used in the second example were transformed using the amino acid z-scores of Sandberg et al. 1998 to substitute for amino acid species, instead of the five physical/chemical properties used in the first two examples. The five z-score values used for each amino acid derive from 10 experimentally determined and 16 calculates physicochemical properties of the amino acids, and are optimised for quantitative sequence-activity modelling. The clustering results using the z-scores were very similar to the results of the second example.

It was observed that human receptors GPR7 and GPR8 clustered in the same cluster as opioid receptors and also close to C-X-C chemokine receptors 3, 4 and 5. In conventional phylogenetic trees, GPR7 and 8 cluster somewhere between opioid receptors and somatostatin receptors and relative far away from chemokine and chemotactic factor receptors.

GPR72 belongs to the same cluster as GPR73, but still has an unknown ligand. Based on a phylogenetic tree and also suggested by Parker et al. 2000 (Biochim. Biophys. Acta 1491:369-375) GPR72 and 73 would be related to neuropeptide receptors. Using the described clustering method, we can deduce that they both might play a role in smooth muscle contraction.

The clustering method places orphan receptors GPR38 and 39 in the vicinity of neuropeptide binding GPCRs. This clustering is consistent with conventional phylogenetic relationships.

1-23. (canceled)

24. A method of clustering a plurality of transmembrane protein sequences, each sequence comprising one or more intra-membrane regions and one or more extra-membrane regions, comprising the steps of:
forming a set of amino acid labels from each protein sequence, each set including a plurality of amino acid labels from one or more of said intra-membrane regions and excluding at least some amino acid labels from said one or more extra-membrane regions, each amino acid label corresponding to a positionally equivalent amino acid label in each of the other sets of amino acid labels;
forming a set of physical/chemical properties from each set of amino acid labels by substituting each amino acid label with one or more physical/chemical properties; and
clustering the sets of physical/chemical properties to thereby identify relationships between said transmembrane protein sequences.

25. The method of claim 24 wherein said physical/chemical amino acid properties are selected from a list comprising: molecular weight, hydrophobicity, hydrophilicity, surface area, acidity and isoelectric point.

26. The method of claim 24 wherein the step of clustering comprises steps of correlating sets of physical/chemical properties for pairs of protein sequences or groups of protein sequences.

27. The method of claim 24 wherein each set of amino acid labels includes substantially all of the amino acid labels from said one or more intra-membrane regions of the corresponding protein sequence.

28. The method of claim 24 wherein each set of amino acid labels excludes substantially all of the amino acid labels from said one or more extra-membrane regions from the corresponding protein sequence.

29. The method of claim 24 wherein the step of forming a set of amino acid labels for each protein sequence comprises the step of carrying out a statistical alignment of said protein sequences to establish the positional equivalence of each of the amino acid labels of each set.

30. The method of claim 24 wherein the transmembrane protein sequences are sequences for G-protein coupled receptors.

31. A method of clustering a plurality of transmembrane protein sequences, comprising the steps of:
isolating equivalent transmembrane domains in each sequence;
substituting the amino acids in each transmembrane domain sequence with one or more physical/chemical properties; and
clustering the resulting sets of physical/chemical properties.

32. The method of claim 31 further comprising the step of displaying textual information relating to each transmembrane protein sequence in an arrangement determined by said step of clustering.

33. The method of claim 31 further comprising the step of inferring a biochemical characteristic of one of said transmembrane proteins from characteristics of others of said transmembrane proteins with which it is clustered.

34. A method of clustering a plurality of polypeptide sequences, comprising the steps of:
forming a set of amino acid labels from each polypeptide sequence, each amino acid label corresponding to a positionally equivalent amino acid label in each of the other sets of amino acid labels;
forming a set of physical/chemical properties from each set of amino acid labels by substituting each amino acid label with one or more physical/chemical property values, and
grouping the sets of physical/chemical properties so as to identify groupings of said polypeptide sequences.

35. Apparatus for clustering a plurality of transmembrane protein sequences, to thereby aid identification of relationships between said transmembrane protein sequences, each sequence comprising one or more intra-membrane regions and one or more extra-membrane regions, comprising:
a segmentor arranged to form a set of amino acid labels from each protein sequence, each set including a plurality of amino acid labels from one or more of said intra-membrane regions and excluding at least some amino acid labels from said one or more extra-membrane regions, each amino acid label corresponding to a positionally equivalent amino acid label in each of the other sets of amino acid labels;
a translator arranged to form a set of physical/chemical properties from each set of amino acid labels by substituting each amino acid label with one or more physical/chemical properties; and
an analyser arranged to cluster or order the sets of physical/chemical properties.

36. The apparatus of claim 35 wherein said physical/chemical amino acid properties are selected from a list comprising: molecular weight, hydrophobicity, hydrophilicity, surface area, acidity and isoelectric point.

37. The apparatus of claim 35 wherein the calculator is adapted to correlate sets of physical/chemical properties for pairs of protein sequences or groups of protein sequences.

38. The apparatus of claim 35 wherein the segmentor is arranged to form each set of amino acid labels to include substantially all of the amino acid labels from said one or more intra-membrane regions of the corresponding protein sequence.

39. The apparatus of claim 35 wherein the segmentor is adapted to form each set of amino acid labels excluding substantially all of the amino acid labels from said one or more extra-membrane regions from the corresponding protein sequence.

40. The apparatus of claim 35 wherein the segmentor is further adapted to carry out a statistical alignment of said protein sequences to establish the positional equivalence of each of the amino acid labels of each set.

41. The apparatus of claim 35 further adapted to present, on a visual display, the sets of physical/chemical properties in a geometry reflecting the results of the clustering effected by the analyser.

42. Apparatus for clustering a plurality of transmembrane protein sequences, comprising:
a segmentor adapted to isolate equivalent transmembrane domains in each sequence;
a translator adapted to substitute the amino acids in each transmembrane domain sequence with one or more physical/chemical properties; and
an analyser adapted to cluster the resulting sets of physical/chemical properties.

43. Apparatus for clustering a plurality of polypeptide sequences, comprising:
a segmentor adapted to form a set of amino acid labels from each polypeptide sequence, each amino acid label corresponding to a positionally equivalent amino acid label in each of the other sets of amino acid labels;
a translator adapted to form a set of physical/chemical properties from each set of amino acid labels by substituting each amino acid label with one or more physical/chemical property values, and
an analyser adapted to group the sets of physical/chemical properties so as to identify groupings of said polypeptide sequences.

44. A computer readable medium carrying computer program elements for clustering a plurality of transmembrane protein sequences to thereby aid identification of relationships between said transmembrane protein sequences, each sequence comprising one or more intra-membrane regions and one or more extra-membrane regions, the program elements comprising:
a segmentor arranged to form a set of amino acid labels from each protein sequence, each set including a plurality of amino acid labels from one or more of said intra-membrane regions and excluding at least some amino acid labels from said one or more extra-membrane regions, each amino acid label corresponding to a positionally equivalent amino acid label in each of the other sets of amino acid labels;
a translator arranged to form a set of physical/chemical properties from each set of amino acid labels by substituting each amino acid label with one or more physical/chemical properties; and
an analyser arranged to cluster or order the sets of physical/chemical properties.

45. A computer program product comprising computer program elements adapted to carry out the steps of claim 44 when executed on a computer system.

46. A computer readable medium comprising computer program elements adapted to carry out the steps of claim 44 when executed on a computer.
